(19) United States
(12) Patent Application Publication
(10) Pub. No.: US 2009/0005840 A1
(43) Pub. Date: Jan. 1, 2009

(54) Anti-shock cooling/heating device

(76) Inventor: Robert D. Harty, Mokena, IL (US)

Correspondence Address: CHERSKOV & FLAYNIK, THE CIVIC OPERA BUILDING, 20 NORTH WACKER DRIVE, SUITE 1447, CHICAGO, IL, 60606, US

(21) Appl. No.: 12/157,448

(22) Filed: Jun. 9, 2008

Related U.S. Application Data
(60) Provisional application No. 60/933,715, filed on Jun. 8, 2007.

(51) Int. Cl. A61F 7/00

(52) U.S. Cl. 607/104

(57) ABSTRACT

A portable device for cooling or heating a body part using readily available cooling and heating sources, such as water from gardening hoses, EMS vehicles, fire trucks, and hydrants is presented. The devices are modular so that multiple devices can be combined to create a large device for cooling or heating an entire body that uses a single cooling or heating source. Alternatively, modularity of the invention provides a means to cool just a specific region of the body while simultaneously warming another region.

### PRIORITY

This utility patent application claims the benefits of U.S. Provisional Patent Application No. 60/933,715, filed on Jun. 8, 2007.

### BACKGROUND OF THE INVENTION

1. Field of the Invention

This invention relates to the field of portable cooling and heating apparatuses, and more specifically, this invention relates to a triage device for the in situ regulation of the temperatures of hypothermic and hyperthermic persons.

2. Background of the Invention

A major impact on firefighters, marathon runners, football players, and many other occupations and sports is fluid loss due to body temperature increases. Often this leads to heat exhaustion, heat stroke, and, in some cases, death.

In a heat related emergency, cooling the surface of a body part is the quickest way to cool the body and control body temperature.

A myriad of patient thermal treatment modalities exist. U.S. Pat. No. 4,729,598 awarded to Hess on Mar. 8, 1988 discloses a convalescent chair. The chair includes a plurality of air sacs through which cool air is pumped to inflate the sacs. However, the present invention is designed to overcome shortcomings of this prior art design. Notably, the Hess chair is not mobile and relies on external electric power. The present invention is fully mobile and can be deployed in any environment, including one that lacks a reliable power supply. Furthermore, contrary to the present invention which can use either liquid or gas, the Hess patent cannot be adapted to use liquids inasmuch as the bladders within the Hess design feature holes to facilitate air discharge. Additionally, the user of the Hess product must remain seated, while the present invention can be adapted for use in both a sitting and prostrate positions. The Hess patent forecloses use of flaps for surrounding fluid contact, nor does Hess suggest use of ice or hot packs in conjunction with the system. The present invention, on the other hand, discloses both flaps which can surround patient extremities, as well as means for receiving hot or cold packs for pinpoint temperature adjustments. The most significant drawback of the Hess device is its reliance on electrical power, especially on its requirement of a powered heat exchanger. Inasmuch as Hess does not function without power, it cannot be used in situations where power is not available. The present invention does not suffer from such a shortcoming.

The use of electric components is common to prior art devices such as U.S. Pat. No. 4,170,998 awarded to Sauder on Oct. 16, 1979, which discloses a cooling sleeve. However, analogously to Hess, the Suader sleeve requires a power source and a bulky powered element (the evaporator) and is completely unsuitable for use in triage situations. The present invention, on the other hand, functions fully without power, nor does it rely on bulky additional modules, ensuring that the present invention can be located at any incident site.

U.S. Pat. No. 5,263,336 awarded to Kuramarohit on Nov. 23, 1993 discloses a cooling suit coat and trousers as well as a cooling chair which may optionally be used with the cooling suit. The present invention overcomes a number of shortcomings in the Kuramarohit system. For example, the Kuramarohit device requires the wearer to don a complete suit, including a jacket with sleeves and trousers. This step is difficult, if not impossible for an unconscious or uncooperative user. The present invention merely requires the user to contact the surface without donning the invention. While Kuramarohit suggests using a cooling chair, it is only in conjunction with the cooling garment. The present invention does not require the user to wear any specific garment, and cooling or heating of the user in the present invention is accomplished regardless of what the user is wearing. A drawback of the Kuramarohit system is that it relies on evaporation of water from the damp cooling suit. This method of cooling is common, but it suffers from drawbacks and as such is not used by the present invention. In the present invention, rather than relying on evaporating cool water, the system transfers heat to or from the user using the cooling or heating fluid in channels of the invented assembly. In some embodiments of the invention, the evaporative method is avoided, while in the Kuramarohit invention the evaporative method is the sole method of cooling.

There are many other ways to cool the body down and replenish it with fluids, particularly in triage situations. Some of these ways include spraying the body with water, applying ice packs, and using fans to reduce one's core temperature, and these are the approaches accepted by Kuramarohit and other prior art systems. Medical researchers have demonstrated for years that the easiest and quickest way to control body temperature is by placing ice packs in strategic places, such as the groin, the back of the neck, under the arm pits, and in one's hand. down with direct contact with water.

There are drawbacks to these methods, however, including the “dampening effect” direct contact of water has on a patient. This dampening effect can result in too rapid chilling, leading to shock. Another drawback of the dampening effect is the advent of steam burns firefighters suffer after they return to fight a fire after cooling.

A need exists in the art for a system to contact large surfaces areas of a hyperthermic or hypothermic patient with cooling substrate or warming substrate, respectively. The system should not dampen the skin of the patient, so as to avoid shock-inducing precipitous drop or rise in temperature. The system should also allow cooling of one body region at the exclusion of other body regions, with those other body regions perhaps being subjected to warming from the same device. The system should be modular so as to allow for multiple devices to be combined so that one large device for cooling or heating an entire body can be used. Additionally, the system should allow for the flow of thermal exchange agents, such as water and air supplied from garden hoses, fluids with high vapor pressure such as various chemical cooling agents (e.g., Freon, ammonia, etc), and various chemical heating agents or those fluids with lower vapor pressure. Such fluids would be transported to the device via common fluid conveyances, conduits, and initially supplied by a myriad of commercial entities.

### SUMMARY OF THE INVENTION

An object of this invention is to provide a portable device for use to cool or warm a human or animal patient's body surface. A feature of the invention is that the device, or pluralities of the device, are deployed to affect regions of the patient's body exclusive of other regions. An advantage of the invention is that blood vessels at one region of a body surface are constricted when cooled by the device, or dilated when warmed by the device, while other regions of the body are reversibly affected or left unaffected.

Another object of this invention is to provide a portable cooling or heating device that can be attached to readily available sources of water and/or pressurized air, such as from ambulances, fire trucks, or other EMS vehicles during an emergency, from standard garden hoses, and from hospitals with air and/or water supply lines. A feature of the device is that it is scalable in that it can be deployed in stages using identical, or similar modules that facilitate interchangeability. A torso-supporting substrate can be employed, then later, a head-supporting substrate, then, or simultaneously, a buttocks supporting substrate. An advantage of the invention is that this modularity confers spot thermal treatment to a patient.

The invention provides a device for cooling or heating a body part, the device comprising a substrate adapted to contact a body part; a fluid transfer system traversing said substrate, said system having a means of fluid ingress and a means of fluid egress; a fluid intake port communicating with said means of ingress and adapted to receive in-flowing fluid; and a fluid discharge port communicating with said means of egress and adapted to evacuate the fluid from the device.

Also provided is a series of n devices, with n being an arbitrary number, for cooling or heating one or more body parts, each said device designated by a consecutive integer l, with l=1 . . . n, each device l comprising a substrate adapted to contact a body part; a fluid transfer system traversing said substrate, said system having a means of fluid ingress and a means of fluid egress; a fluid intake port communicating with said means of ingress and adapted to receive in-flowing fluid; and a fluid discharge port communicating with said means of egress and adapted to evacuate the fluid from the device such that the means of ingress of device i=1 is connected to an external source for the fluid, the means of ingress of each device i=2 . . . n is connected to the means of egress of device i−1, and the means of egress of device n is connected to an external reservoir. Further provided is a plurality of devices interconnected, either in series or parallel, designed to heat or cool multiple users simultaneously. Optionally, the external reservoir is in fluid communication with a recirculating pump that directs the “spent” fluid to a means for reheating, re-cooling, repressurizing or decompressing the temperature exchange fluid (e.g., air, water, Freon, ammonia) prior to reintroduction of the fluid into the device. One such means is a portable cooling apparatus disclosed in U.S. Pat. No. 4,170,998 awarded to Sauder on Oct. 16, 1979 and incorporated herein by reference.

### BRIEF DESCRIPTION OF THE DRAWING

The foregoing and other objects, aspects, and advantages of this invention will be better understood from the following detailed description of the preferred embodiments of the invention with reference to the drawing, in which:

FIG. 1 is an overhead view of a device for cooling or heating a body part, in accordance with features of the present invention;

FIG. 2 is a side view of a device for cooling or heating a body part, in accordance with features of the present invention;

FIG. 3 is an overhead view of a device for cooling or heating a body part with a curved insert for one or two legs, in accordance with features of the present invention;

FIG. 4 is a perspective view of combined devices for cooling or heating a body part (sitting position), in accordance with features of the present invention;

FIG. 5 is a perspective view of combined devices for cooling or heating a body part (flat position), in accordance with features of the present invention;

FIG. 6 is a side view of combined devices for cooling or heating a body part (folded for storage), in accordance with features of the present invention;

FIG. 7 is a plan view of a bladder for accepting thermal treatment fluids, in accordance with features of the present invention;

FIG. 8 is a view of FIG. 7 taken along lines 8-8; and

FIG. 9 is an overhead view of a series of devices for cooling or heating a body part, interconnected in a cascade, in accordance with features of the present invention.

### DETAILED DESCRIPTION OF THE INVENTION

The invention provides for selective heating and/or cooling of body surfaces. One embodiment of the invention provides a means for cooling a first region of the body while a second region of the body is untreated. This is beneficial in instances where blood pooling is to be avoided in an affected area by trauma or medical abnormality but blood flow is to be increased or otherwise encouraged to occur in the remaining portion of the body. Alternatively, the device provides for heating or cooling of a body region while allowing for remainder portions of the body to be unaffected by such thermal treatment. Additionally, the shape and dimensions of the device can be designed for use by newborns, children, adults, and animals.

It should be appreciated that the invention provides a device and method for cooling or warming the blood of a patient in vivo via the application of cool or warm substrates to exterior surfaces of the patient. The invention thermally treats the tissues comprising the surfaces so as to minimize or maximize fluid flow to and from the tissue. Such thermal treatment includes constricting or dilating blood vessels, lymph structures, bone marrow and sinuses defined thereby, and other physiological lumens.

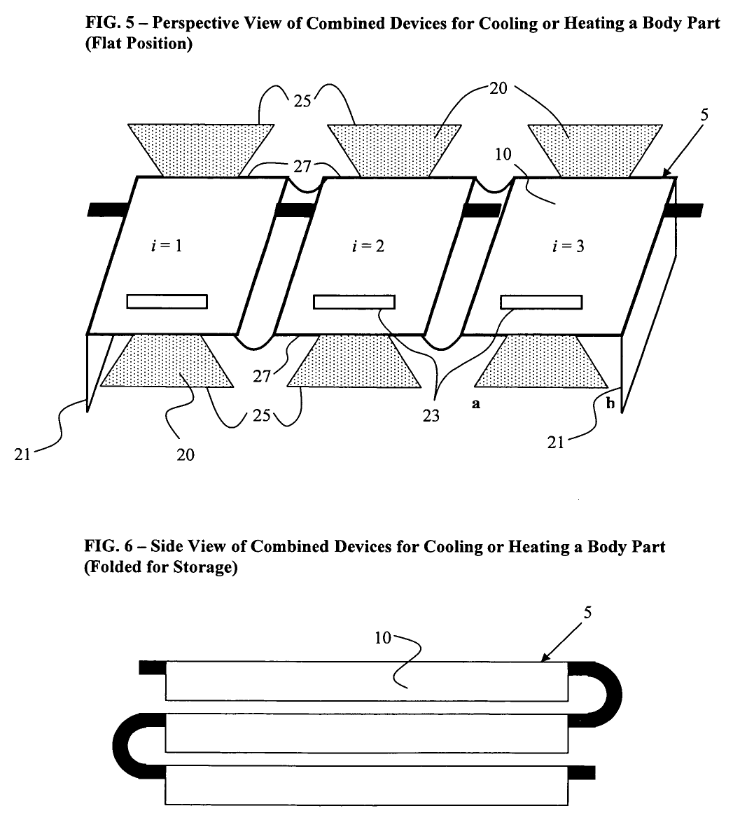

A salient feature of the device is that it can be utilized as a single module, or else integrated with similar modules to encapsulate and effect larger body surfaces. As such, one embodiment of the device is where it constitutes a series arrangement of heat exchangers, which are identical, repeating units in fluid communication with each other so that fluid from a first device passes through to a second, and perhaps a third, device. In other words, a plurality of modules can be connected in series starting from the means of egress of a first module to the means of ingress of a second module situated downstream of the first device. Alternatively, one can state that the present invention provides for a series of n devices, with n being an arbitrary number, for cooling or heating one or more body parts, each said device designated by a consecutive integer i, with i=1, 2, 3, 4 . . . n, each device i comprising: a substrate adapted to contact a body part; a fluid transfer system traversing said substrate, said system having a means of fluid ingress and a means of fluid egress; a fluid intake port communicating with said means of ingress and adapted to receive in-flowing fluid; and a fluid discharge port communicating with said means of egress and adapted to evacuate the fluid from the device; such that the means of ingress of device i=1 is connected to an external source for the fluid, the means of ingress of each device i=2 . . . n is connected to the means of egress of device i−1, and the means of egress of device n is connected to an external reservoir. Such a series arrangement results in a larger fraction of a body's surface being affected by the temperature of the fluid passing through the series of devises. An example of an application of a series arrangement is depicted in FIG. 4, and FIG. 5, discussed more fully infra, wherein three devices are arranged such that a person can sit on several substrates so as to contact an entire side of the person's body to the temperature effect rendered by the device.

Alternative arrangements is whereby one module is a cooling module, while another module, applied to another region of the same patient, is a heating module, so as to effect different heating/cooling regimens to different regions of the same patient.

In the alternative, the modules can be integrated with identical modules in a parallel, as opposed to a series, arrangement.

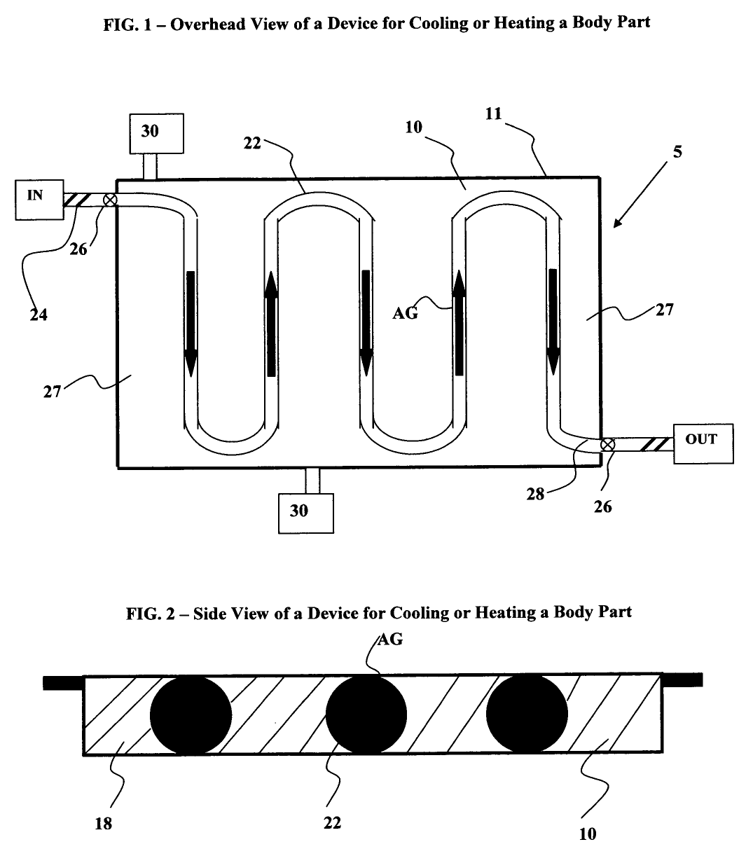

FIG. 1 is an overhead view of a device designated as numeral 5, for cooling or heating a body part. The device includes a reversibly deformable substrate 10. For the sake of specificity, the body part in question is a human upper torso, but the device can be used for other regions of a human or animal body part.

The inside of the substrate 10, as shown in FIG. 2, comprises a plurality of fluid transfer conduits 22, whereby each of the conduits are adapted to receive a variety of fluids. In one embodiment, the conduits are formed of flexible yet noncollapsible material (such as poly vinyl chloride) encapsulated by the reversibly deformable substrate 10 such as a webbing made of flexible foam.

A first end of each of the conduits defines an intake port 24 terminating in a flow control valve 26. The valve 26 is adapted to receive an input conduit which transports fluid to the device.

A second end of the fluid transfer system defines a discharge port 28 terminating in a control valve 26, which is used to start, stop or otherwise regulate fluid evacuation from the system. As such, the control valve 26, in conjunction with a fluid output conduit, defines a means of egress of the fluid from the device.

### Anti-Dampening

### Feature Detail

As discussed supra, a drawback with current cooling and/or warming devices is their proclivity to wet or otherwise dampen the patient. Such dampening can lead to extreme, and rapid swings in temperature. In the case of fire fighters returning to a fire after cool down, water left on the skin or clothing can lead to steam burns.

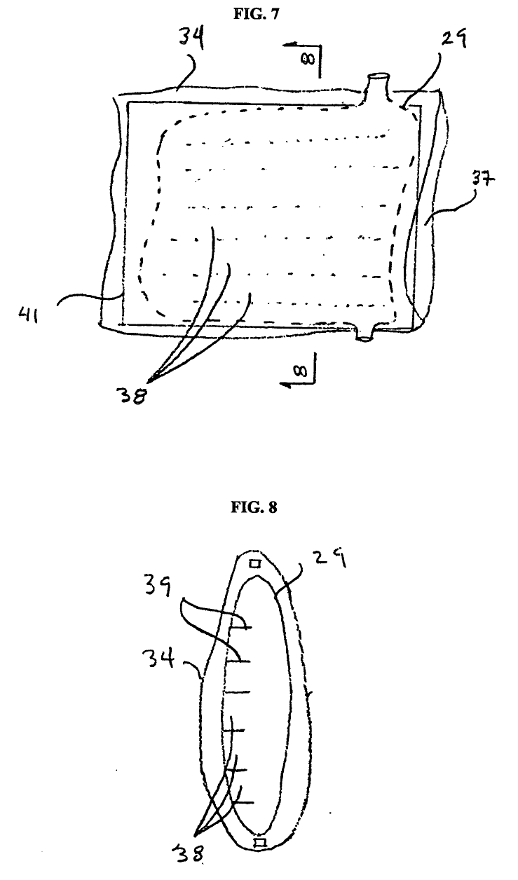

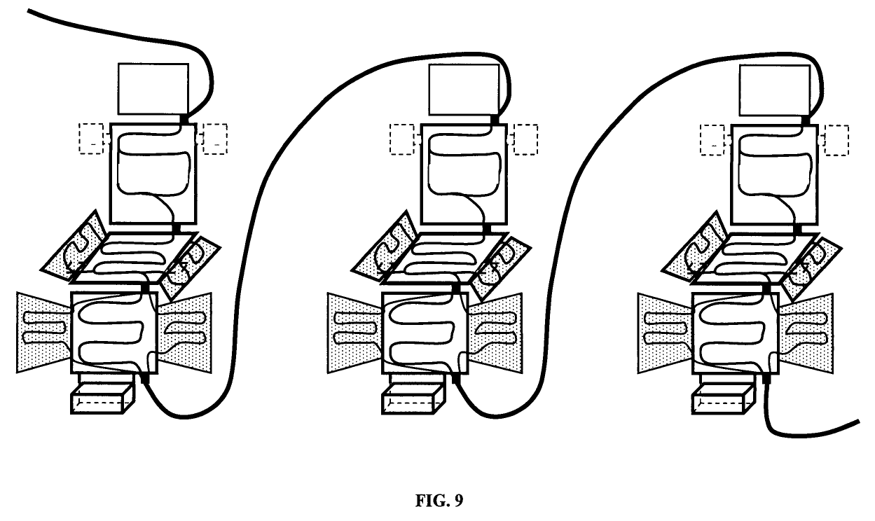

An embodiment of the present invention eliminates the dampening effect by the juxtaposition of a water impermeable webbing between the patient and any conduit through which cooling or warming fluid circulates. In one such embodiment, a fluid reservoir 29 or bladder, reminiscent of a “hot water bottle” is positioned intermediate the intake port 24 and the discharge port 28. Overlaying this bladder, so as to substantially encapsulate it is a water proof liner 34 (see FIG. 7), such that only the liner contacts the patient, and not the encapsulated bladder. The bladder is either removably received by the liner 34 through a reversibly closable opening 37 in the liner or permanently encapsulated by the bladder.

The effect of the liner is to prevent condensation, which forms on the outside surface of the bladder, from contacting and dampening/wetting the patient. Instead, the liner isolates the condensation to the surface of the bladder and prevents transfer of the condensation to the patient.

This reservoir or bladder 29 is adapted to receive, collect and store fluids at various temperatures. In the case of liquids, the reservoirs accumulate liquids at temperatures ranging from their melting points and their boiling points, and as such, the reservoirs are tolerant of such extremes in temperature while maintaining the reversible deformation qualities necessary to impart comfort to the patient. In the case of expanding gases, whereby Freon, ammonia or other high vapor pressure materials are utilized, the fluid transport conduit 22 system is constructed to withstand variations in the pressure introduced by incoming and outflowing fluids. Also, the material is chemical resistant to such high vapor pressure substances.

A means is provided to assure homogenous circulation of cooling and/or warming fluid throughout the bladder. This means is depicted in FIG. 7 as a plurality of channels 38 which are defined by fins 39 extending medially from an inside surface of the bladder along substantially the entire inside surface. The fins are arranged such that their longitudinal axes generally extend parallel to each other. This arrangement provides a means for water to traverse each of the channels, and therefore traverse substantially the entire interior of the bladder, versus simply entering and exiting the bladder via the fluid ingress 24 and egress 28 points by traversing the shortest possible distance through the bladder.

The use of a bladder 29 can be relegated to one pressure surface of the device (for example, the seat back as shown in FIG. 4). Alternatively, a plurality of bladders can be utilized so as to underlie and confer simultaneous temperature change to a plurality of pressure surfaces, such as the buttocks support region “B”, and seat back region “SB”, the leg support region “L”, and the head region “H”. In once embodiment, these regions are connected in a serial fashion such that thermally treated fluid enters one end of the region, for example the foot support region “F”, traverses it, then through the leg-, buttocks-, seat back- and head-supporting regions and exits from the head supporting region. In this embodiment, the entire body is thermally treated with the same fluid so as to be simultaneously cooled. Alternatively, one or more regions of the device are isolated from the traversing fluid such that only select regions, (for example the seat back “SB” and leg supporting regions “L” are in thermal contact with a first fluid, while the remaining regions (such as the intervening buttocks-supporting region “B”) are not in thermal contact with that first fluid, but rather in contact with a second fluid, or in thermal contact with a air-filled bladder, or in thermal contact with a bladder having its internal atmosphere maintained as a vacuum so as to serve as a means for preventing thermal exchange between one fluid-filled region from another.

To confer structure to the various regions, a frame 41 is provided in which the bladder nests. The frame is made of a reversibly deformable material to provide comfort to a patient resting upon it, which also providing a predetermined geometric configuration (such as a square) to define the shape of the support surface. The frame also provides a means for keeping the liner tout so as to confer space between an interior facing surface of the liner and an exterior surface of the bladder. Such space aids in preventing the formation of condensation on the exterior facing surface of the liner.

A periphery 11 of the substrate further defines edges 27 terminating in complementary hook and pile closure means to facilitate removable encirclement of the device around a body part. These edges 27, or alternatively, a plurality of fold-over flexible panels 20 attached to the periphery, feature hook and closure means to allow attachment to each other at their free ends 25. This enables the device to substantially encircle the desired body part. Either arrangement provides a means for wrapping the body part to assure that the part remains immobile and therefore in constant contact with the heat exchange substrate. In instances where complete immobility is essential, an embodiment of the invention features regions of the support substrate 10 defining apertures 23 extending transversely through the substrate and adapted to receive the edges 25 of the flaps. In an embodiment of the invention, the apertures extend in a direction parallel to the line formed by line a-b of the flap's edge (See FIG. 5). This allows a user to first cover the body part (e.g. a leg or arm) with the flap, then extend the edge 25 of the flap through the aperture. The flap is then caused to encircle and anchor to itself via a hook-and-pile connection. This means for wrapping the limb is similar to the configuration of blood pressure cuffs utilized by the same triage personnel. In an embodiment of the device, the flaps are attached to the substrate and so constructed to further serve as a means for restraining an unruly, semi-conscious, or distressed patient.

Each of the fold-over flexible panels 20 can feature circulating fluid (so as to function as a smaller version of the device 5 as depicted in FIG. 1) and therefore part of a serially connected device discussed supra, or else the fold-over flexible panels 20 can be static in design. Such static fold-over flexible panels 20 would further define pockets into which hot or cold packs can be inserted.

Separate from the fold-over panels or the flaps discussed supra, in an embodiment of the device the edges 11 of the substrate 10 as depicted in FIG. 4 are adapted to removably receive pouches 31 in which a patient can place her hands. The pouches are lined with a cold pack, heat pack, or some other means for cooling or warming the hand. Such a pouch features a separate enclosure for the heat/cool pack, and a separate enclosure in which the hand is inserted. The pack enclosure and the hand enclose share a common wall of thin flexible material, which is heat conducting so as to assure heat transfer.

Alternatively, the pouch removably receives the cold pack or heat pack so as to simultaneously accommodate both the pack and the person's hand, such that the person actually directly contacts the pack. Such a pouch is leak proof so that cold water, or loose ice can be accommodated by the pouch, essentially allowing the patient's hand(s) to be immersed directly in the water or ice. Similarly, in another embodiment of the invention, pouches 33 removably attached to a depending edge of a leg-support substrate module of the multi-module device are adapted to receive a foot (one foot per pouch), or feet (both feet in one pouch) of a patient to provide similar cooling/heating.

An embodiment of the invention further comprises a means for hingeably communicating one patient support surface (such as the seat back “SB”) with another patient support surface (such as the buttocks support region “B”). Such means can be a standard piano hinge, or a series of straps extending perpendicular to the junction formed by opposing edges of adjacent support surfaces, whereby the straps are attached to the support surfaces in a hook-and-pile configuration.

To operate the device, or the multi-module device, an operator connects the means of ingress of the device to a warm/cool fluid supply, such as a garden hose, a refrigeration unit, or a warm-fluid supply line. The fluid flow is regulated through the device either by the pressure provided by the fluid supply or by the control valve 26 proximal to the means of ingress. Similarly, the operator of the device can regulate the rate of evacuation of the fluid from the device through manipulation of the control valve that is positioned in close spatial relationship to the means of fluid egress from the device. As noted supra, the “spent” fluid, after exiting the device, is recirculated or retreated to its original temperature, pressure, and or volume. Typical heat exchange units (such as refrigeration units for recooling, or condensation coils for re-heating) are suitable.

The operator positions the device 5 onto the body part either before, during, or after fluid is introduction into the device. Encircling the body part with the device prior to fluid introduction allows the operator to adjust the snugness of the device and therefore adjust the amount of surface area contact of the device to the surface of the patient via manipulation of the control valves 26. The operator may also surround the body part by wrapping the fold-over flexible panels 20 around the body part. When the desired body temperature is achieved, the operator removes the substrate 10 from the body part that is to be cooled or heated, or else adjusts fluid flow so as to maintain a steady state temperature while the device continues to be applied to the patient.

An embodiment of the invention features a downwardly depending leg 21 or plurality of legs in hingeable communication with the substrate 10. This allows for elevating the upper body of a patient in situations involving head injuries, elevating the legs in cases of shock, where it would be desirable for the operator to elevate the victim's feet in order to facilitate the flow of blood back to the victim's heart, or to position the device in a combination of configurations, such as elevating the upper body and lowering the legs in a contour couch arrangement.

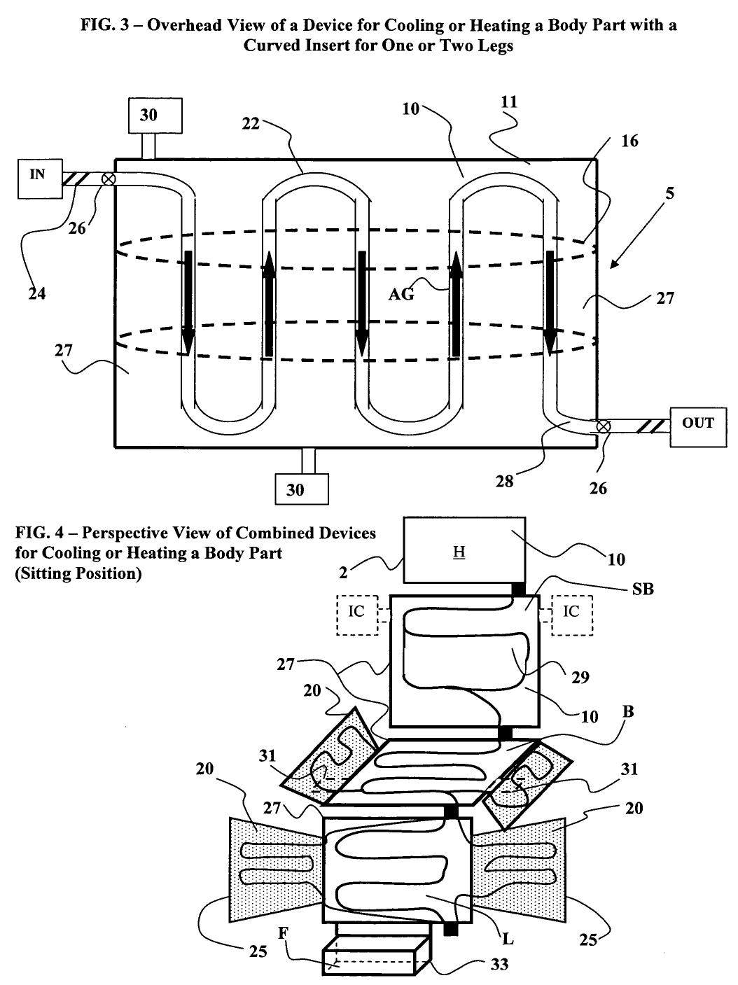

FIG. 3 is an overhead view of a device for cooling or heating a human body part, shaped as a sleeve, in accordance with features of the present invention.

FIG. 4 and FIG. 5 are perspective views of multiple devices in fluid communication with each other, and attached “in series” so as to form a multi-module configuration. The devices as depicted in FIG. 4 and FIG. 5 are instrumental in cooling or heating entire sides of a patient, such as a patient's front or back side (for instance, when the patient is sitting in a chair, laying face up on a gurney, or laying facing down on a gurney). This modular formation is achieved by attaching one device's discharge port 28 with another device's intake port 24, using means such as a standard coupling attachment, thus only requiring one means of ingress or input conduit for an incoming fluid exchange agent and one means of egress for fluid exiting the device.

As depicted in FIG. 5, the coupled devices may be in a flat position to facilitate surrounding of a body part. However, while in the flat position, the combined devices remain separate so as to facilitate elevation of body parts, such as extremities, as is often required in emergency treatment situations. The devices remain interconnected during the elevation.

FIG. 4 depicts a module “H” positioned superior to the remaining modules comprising the multi-module configuration. This superior placement allows module H to accommodate the head of a patient and in one embodiment is adapted to receive temperature exchange fluid coursing through the multi-module configuration. As with the torso-, buttocks- and leg-supporting substrates described supra, edges 27 are adapted to receive flaps 25. However, unlike the torso-, buttocks- and leg-support substrate flaps, the H-module flaps contain regions defining an aperture to allow for access to the ears of the patient if the flaps are deployed about the patient's head. This also allows patients to hear in an unhindered fashion. Another embodiment of module H is as a “static” design and as such is adapted to receive an ice pack, heat pack or the like. Alternatively, in lieu of, or in addition to the substrate 10 comprising module H accommodating ice or heat packs, the aforementioned flaps also can accommodate the ice packs.

In yet another embodiment of the invention, module H removably receives a cervical collar, such as the device claimed in co-owned U.S. patent application Ser. No. 10/633,450 entitled “Integral Head, Neck, and Upper Torso Immobilizer”, and incorporated herein by reference. The cervical collar disclosed in the '450 application is adapted to receive coursing fluid and as such, can be in fluid communication with the instant device.

FIG. 6 is a side view of multiple devices in fluid communication with each other, and attached “in series”, when the devices are folded for storage purposes.

Each device is also structured so that a plurality of devices can be serially connect to cool or heat a plurality of patients, as depicted in FIG. 9 where three devices in a sitting configuration are interconnected together. As shown in FIG. 9, the multiple devices are interconnected in a cascading, or series arrangement. Under this arrangement of n devices, the outlet of the first device is connected to the inlet of the second device until the outlet of the device which is n−1 in the series connects to the inlet of the nth series. Finally the outlet of the nth device is connected to an external outlet. It is further contemplated that the devices can be grouped together in a parallel manner (not depicted), meaning the inlets and outlets of grouped devices are connected to a shared inlet and outlet sources.

The present invention has multiple benefits for use in the medical realm, in addition to cooling and heating a body part. First, this invention is very beneficial for a major trauma patient because the cooling of the body causes vasoconstriction, which increases a patient's blood pressure and helps control blood loss. Second, the fold-over flexible panels 20 are beneficial for applying direct pressure to a body part, thus helping to control the amount of bleeding that takes place for major trauma patients. An embodiment of the invention includes a means for inflating the flaps 20 for further enhancing pressure applied to the limp encircled by the temperature regulated flaps. Third, the fold-over flexible panels 20 are beneficial for redirecting blood out of body areas where blood is not needed, such as the legs, so that the blood is directed into body areas where blood is needed, such as the heart. Fourth, in a mass casualty incident, such as terrorist attacks, this invention can be beneficial for keeping the bodies cool during transport and while awaiting autopsy and embalment procedures. Fifth, the invention is beneficial for certain types of surgical procedures because a mass cooling of the body reduces the need for oxygen and also reduces the amount of blood that flows through the body, therefore allowing the surgery to proceed efficiently and cleanly with less blood loss. Cooling of the patient is done routinely in neurosurgery. Sixth, the invention is beneficial for reducing and healing bed sores of bed ridden patients by allowing one to change the amount and rate of flow of fluid through the device. In this instance, the device is adapted to accommodate pulsating fluid flow, including momentary surges or even cessation of fluid flow through its fluid transport conduit 22 network. Such cessation/surging feature is applicable to treat a myriad of medical conditions. Specifically, such feature helps the healing of bed sores of bed-ridden patients and/or to jostle sleep apnea patients back to a normal breathing pattern in instances where breathing stops or decreases to a dangerous level.

Additionally, the invention is beneficial for multiple scenarios when it is attached to hospital and EMS (Emergency Medical Services) gurneys and backboards, including: keeping newborns warm; keeping febrile children cool; keeping hypothermic patients warm; instituting controlled warming of cold water drowning victims; keeping hyperthermic patients cool; and keeping high fever patients cool.

Finally, the invention has several recreational applications, including cooling or warming of persons reclining in lawn chairs in the summer or fall, cooling or warming of over-the-road truckers, and cooling or warming of long haul aviators, ship pilots, and mobile home retirees. Inasmuch as the materials used to manufacture embodiments of the bladders incorporated in the device are selected to have burst strengths in excess of 10,000 pounds, these myriad of uses of the device are accommodated. The only limitation to the construction of the device is that substantially all of the materials should be radiolucent to afford simultaneous thermal treatment of a body part while affording x-ray access to the body part.

While the invention has been described in the foregoing with reference to details of the illustrated embodiments, these details are not intended to limit the scope of the invention as defined in the appended claims.

The embodiment of the invention in which an exclusive property or privilege is claimed is defined as follows.

1. A device for cooling or heating a body part comprising:
a) a substrate adapted to contact the body part;
b) a fluid transfer system traversing said substrate, said system having a means of fluid ingress and a means of fluid egress;
c) a fluid intake port communicating with said means of ingress and adapted to receive in-flowing fluid; and
d) a fluid discharge port communicating with said means of egress and adapted to evacuate the fluid from the device.

2. The device as recited in claim 1, wherein the body part is a human body part and the substrate is adapted to substantially contact the human body part.

3. The device as recited in claim 1, further comprising a fluid reservoir positioned intermediate the fluid intake port and the fluid discharge port.

4. The device as recited in claim 1, wherein the substrate further comprises: a reversibly deformable material that encapsulates the fluid transfer system.

5. The device as recited in claim 1, wherein the substrate further comprises: a means to attach cooling or heating packs in one or more external pockets.

6. The device as recited in claim 1, wherein the substrate further comprises: a plurality of fold-over flexible panels adapted to surround a body part and further comprising a means to attach the fold-over flexible panels to one another and to regions of the substrate.

7. The device as recited in claim 1, wherein the substrate further comprises: a fold-down elevation grid adapted to elevate a body part at multiple angles.

8. The device as recited in claim 1, wherein the tubular transfer system further comprises a flexible material surrounded by a reversibly deformable envelope.

9. The device as recited in claim 1, wherein the intake port further comprises: a control valve in communication with the means of ingress and adapted to control the amount of fluid entering the fluid transfer system.

10. The device as recited in claim 1, wherein the discharge port further comprises: a control valve in communication with the means of egress and adapted to control the amount of fluid exiting the device.

11. The device as recited in claim 1, wherein the fluid is a liquid.

12. The device as recited in claim 1, wherein the fluid is a gas.

13. The device as recited in claim 1, wherein a series of the devices can be connected from the means of egress of one device to the means of ingress of another device.

14. The device as recited in claim 1 further comprising a means for keeping the body part dry, the means comprising:
a) a bladder integrally molded with the fluid transfer system; and
b) a water proof webbing overlying the bladder.

15. The device as recited in claim 14 wherein an interior surface of the bladder defines a plurality of fins arranged parallel to each other so as to direct fluid entering the bladder to traverse substantially the entire interior of the bladder before exiting the bladder.

16. The device as recited in claim 14 wherein the webbing removably receives the bladder.

17. A series of n devices, with n being an arbitrary number, for cooling or heating one or more body parts, each said device designated by a consecutive integer i, with i=1 . . . n, each device i comprising:
a) a substrate adapted to contact a body part;
b) a fluid transfer system traversing said substrate, said system having a means of fluid ingress and a means of fluid egress;
c) a fluid intake port communicating with said means of ingress and adapted to receive in-flowing fluid; and
d) a fluid discharge port communicating with said means of egress and adapted to evacuate the fluid from the device
such that the means of ingress of device i=1 is connected to an external source for the fluid, the means of ingress of each device i=2 . . . n is connected to the means of egress of device i−1, and the means of egress of device n is connected to an external reservoir.

18. A series of n devices as recited in claim 17 wherein the n devices are interconnected in series to simultaneously heat or cool a plurality of users.

19. A series of n devices as recited in claim 17 wherein the n devices are interconnected in parallel to simultaneously heat or cool a plurality of users.
